(19) United States
(12) Patent Application Publication
(10) Pub. No.: US 2020/0286224 A1
(43) Pub. Date: Sep. 10, 2020

(54) CT LUNG ELASTOGRAPHY WITH A VENTILATION ASSIST SYSTEM

(71) Applicant: KONINKLIJKE PHILIPS N.V., EINDHOVEN (NL)

(72) Inventors: MICHAEL GRASS, BUCHHOLZ IN DER NORDHEIDE (DE); THOMAS KOEHLER, NORDERSTEDT (DE); JOACHIM KAHLERT, AACHEN (DE); JÖRG SABCZYNSKI, NORDERSTEDT (DE); SVEN KABUS, HAMBURG (DE)

(21) Appl. No.: 16/649,432

(22) PCT Filed: Sep. 20, 2018
(86) PCT No.: PCT/EP2018/075546

§ 371 Date: Mar. 20, 2020

Related U.S. Application Data
(60) Provisional application No. 62/561,240, filed on Sep. 21, 2017.

(51) Int. Cl. G06T 7/00; A61B 5/00; A61B 5/085; G06T 11/00; G06T 5/00; G06T 3/40

(52) U.S. Cl. CPC G06T 7/0012; A61B 5/0036; A61B 5/085; G06T 11/003; G06T 2211/424; G06T 3/4076; G06T 2207/20201; G06T 2207/30061; G06T 2207/10081; G06T 5/003

(57) ABSTRACT

A system (100) includes an imaging system (102) and a pressure delivery system (104). The imaging system includes a data acquisition system (114 and 116) and is configured to produce data. The pressure delivery system is configured to produce a periodic airflow variation. The system further includes an operator console (120) configured to control the imaging system to scan a subject receiving the periodic airflow variation and map the periodic airflow variation and first data. The system further includes a reconstructor (516) configured to reconstruct the first data and generate first volumetric image data indicative of the periodic airflow variation.

### FIELD OF THE INVENTION

The following generally relates to imaging and more particularly to computed tomography (CT) lung elastography with a ventilation assist system.

### BACKGROUND OF THE INVENTION

Forced oscillation technique (FOT) and impulse oscillometry systems (IOS) are techniques for functional lung assessment, e.g., to assess lung disease such as chronic obstructive pulmonary disease (COPD) and idiopathic pulmonary fibrosis (IPF). These approaches measure lung function using a pressure wave oscillation generated by a loud speaker and superimposed over tidal breathing or forced over a breath hold. The output is a measure of ventilation over the entire oscillation. Unfortunately, the output is a measure of the response integrated over the entire respiratory system and thus provides no spatial (depth) resolution. That is, lower frequency oscillations penetrate deeper than higher frequency oscillations, and the output does not resolve the depth information.

One approach to resolve the depth information is to apply FOT several times, each with an oscillation having a predetermined frequency, which is different from the other oscillation frequencies. This provides spectral (frequency) information. The output at higher frequencies are used to estimate lung function at greater depths such as in the alveoli and bronchiole and other deeper tissue. The output at lower frequencies are used to estimate lung function at shallower depths such as in the trachea and primary bronchi and other shallower tissue, and output at frequencies there between are used to estimate lung function at depths in tissue there between. Unfortunately, these are only estimates and the measurements still lack spatial resolution.

A computed tomography (CT) scanner generally includes an x-ray tube mounted on a rotatable gantry opposite one or more rows of detectors. The x-ray tube rotates around an examination region located between the x-ray tube and the one or more rows of detectors and emits radiation that traverses the examination region and a subject and/or object disposed in the examination region. The one or more rows of detectors detect radiation that traverses the examination region and generate a signal indicative of the examination region, which is reconstructed to generate one or more images. The literature indicates lung elasticity has been estimated by registering two CT images, one acquired during inhale and the other acquired during exhale, with the result used to assess the COPD stage.

### SUMMARY OF THE INVENTION

Aspects described herein address the above-referenced problems and others.

In one aspect, a system includes an imaging system and a pressure delivery system. The imaging system includes a data acquisition system and is configured to produce first imaging data. The pressure delivery system is configured to produce a periodic airflow variation. The system further includes an operator console configured to control the imaging system to scan a subject receiving the periodic airflow variation and map the periodic airflow variation and first imaging data. The system further includes a reconstructor configured to reconstruct the first imaging data and generate first volumetric image data indicative of a response to the periodic airflow variation.

In another aspect, a computer readable medium is encoded with computer executable instructions, which, when executed by a processor of a computer, cause the processor to: receive characteristics of a periodic airflow variation induced during a scan of a subject with an imaging system, receive imaging data generated by the imaging system with data acquired during the induced periodic airflow variation, correlate the characteristics and the imaging data as a function of time, and reconstruct the imaging data and generate first volumetric image data indicative of a response to the periodic airflow variation.

In another aspect, a method includes receiving, from a pressure delivery system, a frequency and an amplitude of periodic airflow variation induced by the pressure delivery system during a scan of a subject with an imaging system. The method further includes receiving, from the imaging system, imaging data generated by the imaging system with data acquired during the induced periodic airflow variation. The method further includes associating, with a processor, the characteristics and angular views of the data. The method further includes reconstructing, with a reconstructor, imaging data and generating first volumetric image data indicative of a response to the periodic airflow variation.

### BRIEF DESCRIPTION OF THE DRAWINGS

The invention may take form in various components and arrangements of components, and in various steps and arrangements of steps. The drawings are only for purposes of illustrating the preferred embodiments and are not to be construed as limiting the invention.

FIG. 1 schematically illustrates a system including an imaging system and a pressure delivery system.

FIG. 2 schematically illustrates an example of the pressure delivery system.

FIG. 3 schematically illustrates the imaging system supporting a subject in connection with scanning while inducing a forced oscillation with the pressure delivery system.

FIG. 4 graphically illustrates projection data for different phases of the forced oscillation.

FIG. 5 illustrates another example method in accordance with an embodiment herein.

### DETAILED DESCRIPTION OF EMBODIMENTS

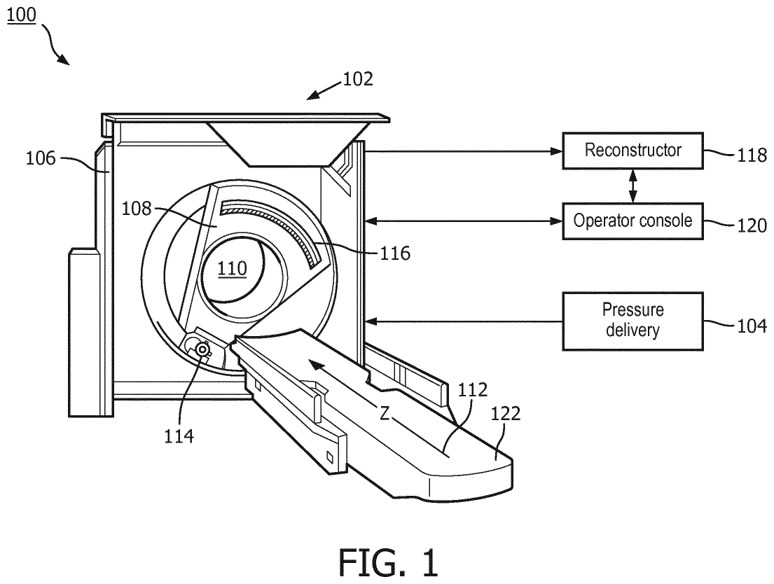

FIG. 1 schematically illustrates a system 100 including an imaging system 102, such as a computed tomography (CT) scanner, and a pressure (e.g., sound, air, etc.) delivery system 104. The imaging system 102 includes a generally stationary gantry 106 and a rotating gantry 108. The rotating gantry 108 is rotatably supported by the stationary gantry 106 and rotates around an examination region 110 about a longitudinal or z-axis 112. A subject support 122 supports an object or subject in the examination region 110.

A radiation source 114, such as an x-ray tube, is rotatably supported by the rotating gantry 108, rotates with the rotating gantry 108, and emits X-ray radiation that traverses the examination region 110. A one or two-dimensional radiation sensitive detector array 116 subtends an angular arc opposite the radiation source 114 across the examination region 110, detects radiation traversing the examination region 110, and generates projection data (i.e. line integrals) indicative of the detected radiation. Collectively, the radiation source 114 and the detector array 116 are referred to herein as a data acquisition system.

The pressure delivery system 104 includes a FOT, IOS, biphasic positive airway pressure (BiPAP) and/or continuous positive airway pressure (CPAP) device, a mechanical ventilator such as a breathing mask, etc., and is employed to induce pressure and/or volume oscillations during a lung scan(s). A reconstructor 118 reconstructs regional lung tissue elasticity, a mean tissue displacement and/or a maximum tissue displacement in different phases of the oscillation and/or relative to a static image based on the oscillations in relation to the data acquisition frequency.

An operator console 120 includes an output device(s) such as a display monitor, a filmer, etc., and an input device(s) such as a mouse, keyboard, etc. The operator console 120 allows an operator to interact with the system 100. This includes selecting an imaging acquisition protocol (e.g., lung scan with induced pressure oscillation), selecting a reconstruction (e.g., elastography) algorithm, invoking scanning, etc. This also includes receiving and recording oscillation characteristics (e.g., frequency and/or amplitude) and/or the ventilation measurement from the pressure delivery system 104.

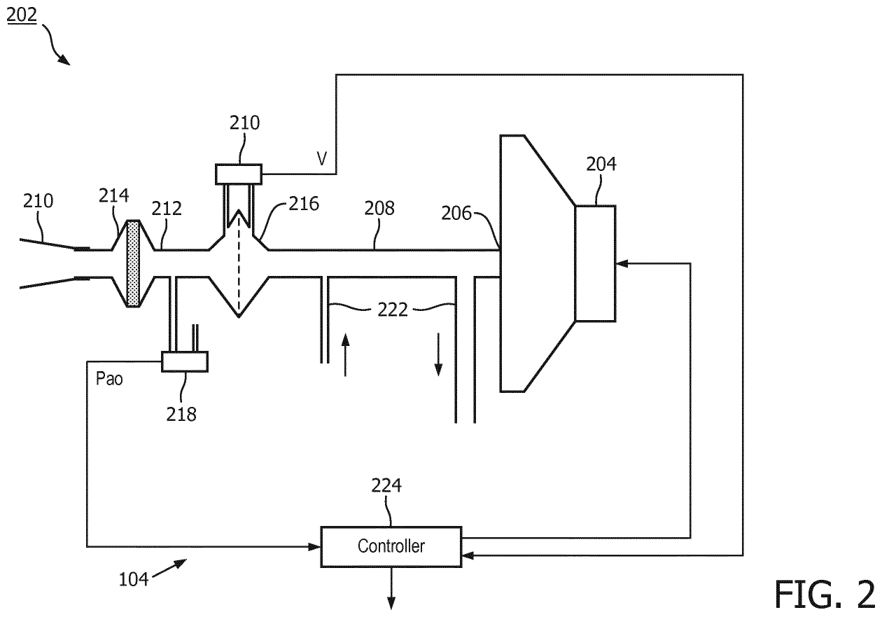

FIGS. 2-4 describe an example where the pressure delivery system 104 includes a FOT device 202.

In FIG. 2, the FOT device 202 includes a loudspeaker 204 mechanically connected to a first end 206 of an elongate hollow tube 208, and a mouth piece 210 mechanically connected to a second opposing end 212 of the tube 208. The illustrated mouth piece 210 includes a bacterial filter 214. The tube 208 includes a pneumatochograph 216. A first transducer 218 is disposed between the filter 214 and the pneumatochograph 216 and is configured to measure pressure (Pao). A second transducer 220 is disposed at the pneumatochograph 216 and is configured to measure flow (V′). Channels 222 are disposed between the loudspeaker 204 and the pneumatochograph 216 and can be used to flush dead space. In a variation, the filter 214 and/or channels 222 can be omitted.

A controller 224 generates and transmits an excitation signal. The excitation signal is an electrical control signal that drives the loudspeaker 204 to produce a pressure oscillation having a predetermined frequency and amplitude. The excitation signal can be preprogrammed, a default algorithm(s), user specified, and/or otherwise determined. The loudspeaker 204 receives the excitation signal and, in response thereto, produces the pressure oscillation. By way of non-limiting example, in one instance the excitation signal results in the loudspeaker 204 generating a pressure oscillation having a given frequency above the normal breathing cycle (e.g., 10-20 Hz) and an amplitude (e.g., 1 cmH₂O) of interest. The pressure oscillation is conveyed to lungs of a subject via the tube 208 and the mouth piece 210.

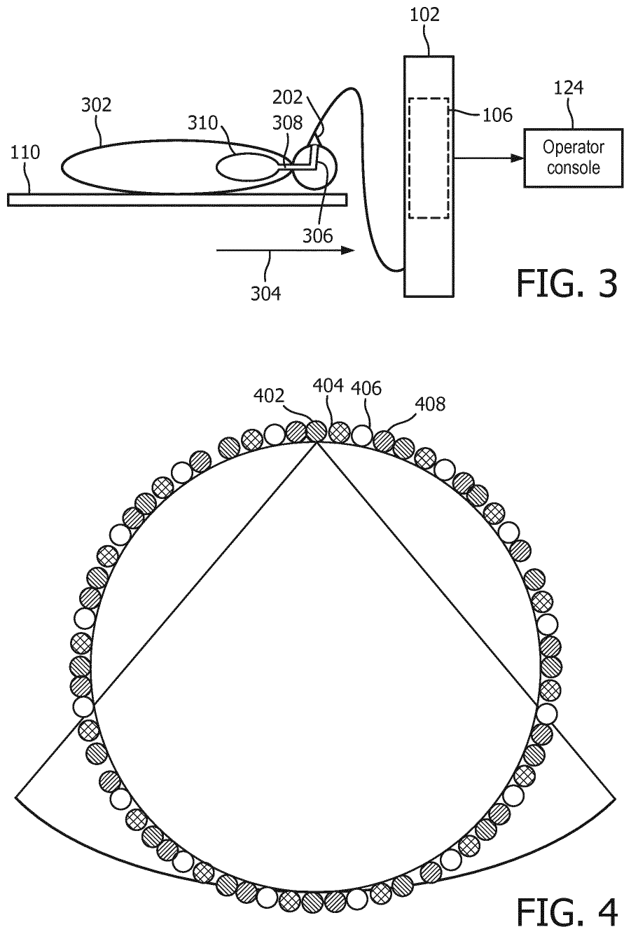

FIG. 3 shows a subject 302 supported by the subject support 122 and moving 304 into the examiner region 110 for a scan. The mouth piece 210 (FIG. 2) of the FOT device 202 is at a mouth 306 of the subject 302, and the pressure oscillations are propagated from the mouth piece 210 (FIG. 2) and through the mouth 306 and a trachea 308 to lungs 310 of the subject 302. The pressure oscillation (e.g., a forced sinusoidal variation of airflow) causes the lungs 310 to expand and contract, based on its frequency and amplitude, during scanning. As such, the subject 302 is scanned as the lungs 310 are induced to expand and contract. The subject 302 can also be scanned without a pressure oscillation, e.g., with the FOT device 202 inactive, not producing a pressure oscillation and/or removed from the subject 302.

With reference to FIGS. 1-3, the controller 224 conveys the oscillation (predetermined frequency and amplitude) information to the console 120 via the stationary gantry 106 (as illustrated in FIGS. 1 and 3) and/or directly thereto. The console 120 correlates the pressure oscillations with data acquisition (projection data). For example, the console 120 maps the different phases of the oscillation with the rotation time so that the projection data (acquisition views) for a particular phase of interest can be extracted and reconstructed to generate volumetric image data for that particular phase. In one instance, the projection data is acquired on an order of ten kilohertz (10 kHz), and images are generated on an order of four (4) Hz.

The following describes example approaches for reconstructing images which contain information about tissue elasticity.

In one instance, a single lung scan is performed with induced oscillations and projection data is generated and reconstructed to produce an image of the lung. With a cycle length of fifty milliseconds (50 ms) and a rotating gantry 108 with rotation times of two (2) seconds, there are approximately 40 oscillations during one rotation. With a projection acquisition rate of 2 kHz, there are 4000 projections per turn and 100 projections in each oscillation. From this, the reconstructor 118 can reconstruct 100 images from 40 projections each from a single turn, or when performing temporal grouping (binning) of projections (e.g. always 25 neighboring ones), 4 images per turn from 4 different time points during the oscillation. FIG. 4 show a repeating pattern of 4 different time points 402, 404, 406 and 408 during oscillation. In this example, the different views of projection data are sorted according to oscillation phase. The projection data for each phase can then be reconstructed to generate volumetric image data for each phase.

From this projection data, the reconstructor 118 can reconstruct a deformation induced by the FOT and an absorption coefficient at a same time (concurrently) using an iterative reconstruction algorithm. In this example, a total amount of projection data is reduced by a factor of five (5) for each phase image. However, images can still be reconstructed and analyzed. This can be achieved by reconstructing a sparse image, applying an inverse sparsifying transform to transform the sparse image back to a target image. An example of this approach is discussed in Chen et al., “Prior image constrained compressed sensing (PICCS): A method to accurately reconstruct dynamic CT images from highly under sampled projection data sets,” Med. Phys. 35 (2), February 2008, 660-663. Other approaches are also contemplated herein.

From this same projection data, alternatively, or additionally, the reconstructor 118 reconstructs a single high-resolution image from the oscillation phase images using a motion compensated reconstruction algorithm. For this, a motion vector field can be determined from an uncompensated image data set. Then, surface models of the lung and the ribs are tracked through the data set to create motion information within the thorax. Then, an image is reconstructed using motion compensated back-projection. An example reconstruction algorithm is discussed in Köhler et al., “Correction of Breathing Motion in the Thorax for Helical C T,” TSINGHUA SCIENCE AND TECHNOLOGY, pp 87-95, Volume 15, Number 1, February 2010. Other approaches are also contemplated herein.

Alternatively, or additionally, two scans are performed. First projection data is acquired without any induced oscillations and a first image of the lung is reconstructed from the first projection data. The pressure delivery system 104 is then utilized to induce oscillations, and second projection data is acquired concurrently with the induced oscillations and a second image of the lung is reconstructed from the second projection data. The second image is a blurred image, e.g., due to the motion from the induced oscillations. The first image can be blurred to match the second image, based on the frequency of the oscillations and/or otherwise. A local amplitude can be estimated using an optimization scheme. This can be applied to a single scan or multiple different scans with varying excitation frequency and/or amplitude.

For blurring, in one instance, a Gaussian low pass filter can be applied locally to the first image (e.g., to patches or sub-regions such as 32×32 regions of a 512×512 image). Alternatively, the Gaussian low pass filter can be applied globally to the first image (i.e. to the entire first image). A width of the filter kernel is such that the blur in the blurred image matches the blur in the FOT image. In one instance, the kernel is selected, e.g., to maximize a similarity measure, such as a cross-correlation and/or other measure of similarity, between the blurred image and the FOT image. An example of this approach for matching resolution between images is discussed in Liow et al., “The convergence of object dependent resolution in maximum likelihood based tomographic image reconstruction,” Phys. Med. Biol. 38 (1993) 55-70. Other approaches are also contemplated herein.

Alternatively, or additionally, the projection data required to reconstruct one oscillation phase image are subdivided into angular segments, and the acquisition and FOT frequency are optimized such that the different angular segments sum up to the total angular range required for reconstruction for every oscillation phase and image slice. For this, the amount of data taken from a preselected phase is adjusted such that at least a predetermined amount is guaranteed for every voxel in the reconstruction volume. This is based on the data completeness requirement that every voxel needs to receive a sufficient illumination required for image reconstruction (e.g, for a 2D image the data for 180°+fan-angle, and for a 3D volume the first and last ray of the data need to be diametrically opposed). An example is discussed in Manzke et al., “Temporal resolution optimization in cardiac cone beam CT,” Med. Phys. 30 (12), December 2003, 3072-3080. Other approaches are also contemplated herein.

In the above example, the pressure delivery system 104 transmits an oscillation frequency and an amplitude of the variation to the console 120. Alternatively, or additionally, the console is configured to produce a sinogram from the projection data and determine an oscillation frequency and an amplitude of the variation from an analysis and/or evaluation of full or on a region of interest of the sinogram.

By way of non-limiting example, a point in the 3D image space is projected due to the well-defined acquisition geometry of the CT scanner on a known sinusoidal trajectory in the sinogram. All the known trajectories of object points in the sinogram will be modified by an additional oscillation which represents the oscillation induced by the pressure delivery system. Frequency analysis along the sinusoidal trajectories in the sinogram will deliver the frequency of the oscillation and the projected amplitude. Effects due to induced displacements along the projection direction of the ray will lead to a rotation angle dependent detectability of frequency and amplitude. In order to increase the detectability of the induced oscillations in the sinogram, the organ of interest (lung) may be segmented from the data set prior to frequency analysis in the sonogram. Segmentation of the non-lung area, forward projection and subtraction from the original sinogram will lead to a region of interest sonogram with better detectability.

Whether oscillating the lungs or other organ, the pressure delivery system 104 is not in the examination region 110 (i.e. not in a field of view therein) and thus does not induce artefacts in the projection data and/or reconstructed image.

The approach(s) described here may be combined with (non-spectral) CT, spectral (multi-energy) CT, phase contrast CT, and/or a different tomographic imaging device such a magnetic resonance imaging (MRI), X-ray tomography, etc.

Although the above example is explained in the context of the FOT device 202, it is to be understood that the dynamic airflow variation can be performed by an IOS, BIPAP, mechanical ventilator and/or other device.

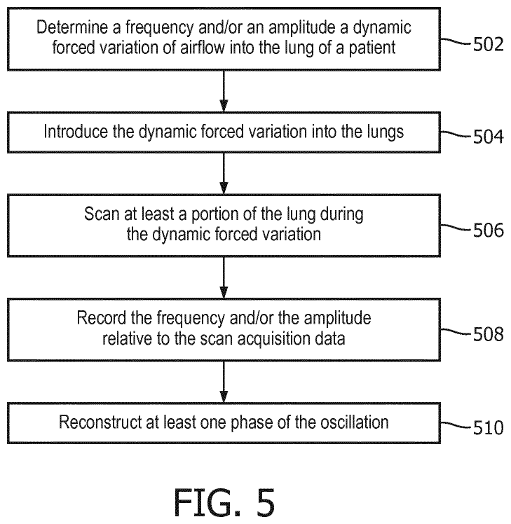

FIG. 5 illustrates an example method in accordance with an embodiment(s) described herein.

It is to be appreciated that the ordering of the acts in the method is not limiting. As such, other orderings are contemplated herein. In addition, one or more acts may be omitted and/or one or more additional acts may be included.

At 502 a frequency and/or an amplitude a dynamic forced variation of airflow into the lung of a patient are determined, as described herein and/or otherwise.

At 504, the dynamic forced variation (an airflow oscillation) is introduced into the lungs, as described herein and/or otherwise.

At 506, concurrently, at least a portion of the lung is scanned, as described herein and/or otherwise.

At 508, concurrently, the frequency and/or the amplitude is recorded relative to the scan acquisition data, as described herein and/or otherwise.

At 510, the acquisition data is reconstructed for at least one phase of the oscillation, as described herein and/or otherwise.

The above may be implemented by way of computer readable instructions, encoded or embedded on computer readable storage medium, which, when executed by a computer processor(s), cause the processor(s) to carry out the described acts. Additionally, or alternatively, at least one of the computer readable instructions is carried by a signal, carrier wave or other transitory medium, which is not computer readable storage medium.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims.

In the claims, the word “comprising” does not exclude other elements or steps, and the indefinite article “a” or “an” does not exclude a plurality. A single processor or other unit may fulfill the functions of several items recited in the claims. The mere fact that certain measures are recited in mutually different dependent claims does not indicate that a combination of these measured cannot be used to advantage.

A computer program may be stored/distributed on a suitable medium, such as an optical storage medium or a solid-state medium supplied together with or as part of other hardware, but may also be distributed in other forms, such as via the Internet or other wired or wireless telecommunication systems. Any reference signs in the claims should not be construed as limiting the scope.

1. A system, comprising:
an imaging system with a data acquisition system configured to produce first data;
a pressure delivery system configured to produce a periodic airflow variation;
an operator console configured to control the imaging system to scan a subject receiving the periodic airflow variation and map the periodic airflow variation and the first data; and
a reconstructor configured to reconstruct the first data and generate first volumetric image data indicative of a response to the periodic airflow variation.

2. The system of claim 1, wherein the pressure delivery system is further configured transmit a frequency and an amplitude of the periodic airflow variation to the operator console, which maps the frequency and the amplitude of the periodic airflow variation with a rotation time.

3. The system of claim 1, wherein the console is further configured to produce a sinogram from the first data and determine a frequency and an amplitude of the periodic airflow variation from the sinogram.

4. The system of claim 1, wherein the first volumetric image data includes voxels with information representing a static image based on the periodic airflow variation in relation to a data acquisition frequency.

5. The system of claim 1, wherein the operator console is further configured to control the imaging system to scan the subject without the periodic airflow variation, generate second data, and the reconstructor is further configured to reconstruct the second data and generate second volumetric image data, and blur the second volumetric image data to match a blur of the first volumetric image data.

6. The system of claim 5, wherein the reconstructor is further configured to determine a local amplitude from the blurred second volumetric image data and the first volumetric image data.

7. The system of claim 5, wherein the reconstructor is further configured to concurrently reconstruct a deformation induced by the periodic airflow variation and an absorption coefficient using an iterative reconstruction algorithm.

8. The system of claim 1, wherein the reconstructor is further configured to sort the first data into a plurality of sub-sets, each corresponding to a different phase of the variation, and reconstruct a sub-set corresponding to a phase of interest and generate second first volumetric image data for the phase of interest.

9. The system of claim 8, wherein the reconstructor is further configured to reconstruct a single high-resolution image from the plurality of sub-sets using a motion compensated reconstruction algorithm.

10. The system of claim 8, wherein the reconstructor is further configured to subdivide a sub-set of the plurality of sub-sets into angular segments such that the different angular segments sum up to a total angular range required for reconstruction for every phase.

11. The system of claim 1, wherein the pressure delivery system includes at least one of a forced oscillation technique device, an impulse oscillometry system device, a biphasic positive airway pressure device, and a continuous positive airway pressure device.

12. The system of claim 1, wherein the imaging system includes a computed tomography scanner.

13. A non-transitory computer readable medium encoded with computer executable instructions, which, when executed by a processor of a computer, cause the processor to:
receive periodic airflow variation characteristics of a periodic airflow variation induced during a scan of a subject with an imaging system;
receive imaging data generated by the imaging system with data acquired during the induced periodic airflow variation;
correlate the periodic airflow variation characteristics and the imaging data as a function of time; and
reconstruct first imaging data and generate first volumetric image data indicative of a response to the periodic airflow variation.

14. The non-transitory computer readable medium of claim 13, wherein the periodic airflow variation characteristics include a frequency and an amplitude of the periodic airflow variation.

15. The non-transitory computer readable medium of claim 13, wherein the computer executable instructions, when executed by the processor, further cause the processor to:
determine at least one of regional lung tissue elasticity, a maximum tissue displacement in different phases of the periodic airflow variation and a static image based on the periodic airflow variation in relation to a data acquisition frequency.

16. The non-transitory computer readable medium of claim 13, wherein the computer executable instructions, when executed by the processor, further cause the processor to:
reconstruct a sub-set of the first volumetric image data corresponding to a phase of interest of a plurality of different phases of the periodic airflow variation and generate second first volumetric image data for the phase of interest.

17. A method, comprising:
receiving, from a pressure delivery system, a frequency and an amplitude of a periodic airflow variation corresponding to a periodic airflow variation induced by the pressure delivery system during a scan of a subject with an imaging system;
receiving, from the imaging system, imaging data generated by the imaging system with data acquired during the induced periodic airflow variation;
associating, with a processor, the periodic airflow variation characteristics and angular views of the imaging data; and
reconstructing, with a reconstructor, imaging data and generating first volumetric image data for the periodic airflow variation.

18. The method of claim 17, further comprising:
determining at least one of regional lung tissue elasticity, a maximum tissue displacement in different phases of the periodic airflow variation and a static image based on the periodic airflow variation in relation to a data acquisition frequency.

19. The method of claim 17, further comprising:
reconstructing a sub-set of the first volumetric image data corresponding to a phase of interest of a plurality of different phases of the periodic airflow variation and generate second first volumetric image data for the phase of interest.

20. The system of claim 17, further comprising:
controlling the pressure delivery system to deliver the periodic airflow variation during the scan of the subject with an imaging system.
